(19) United States
(12) Patent Application Publication
(10) Pub. No.: US 2008/0065126 A1
(43) Pub. Date: Mar. 13, 2008

(54) Excision instrument

(75) Inventor: Masahiro Endo, Seki-shi (JP)

Correspondence Address: POSZ LAW GROUP, PLC, 12040 SOUTH LAKES DRIVE, SUITE 101, RESTON, VA, 20191, US

(73) Assignee: KAI R&D CENTER CO., LTD., Gifu-ken (JP)

(21) Appl. No.: 11/896,809

(22) Filed: Sep. 6, 2007

(30) Foreign Application Priority Data
Sep. 8, 2006 (JP) 2006-243918

(51) Int. Cl. A61B 17/32; B26B 9/00

(52) U.S. Cl. 606/167; 30/346

(57) ABSTRACT

An excision instrument includes a blade 2 mounted on the back surface of an elastic member 5 provided between gripping extensions 3, and is used by bringing the gripping extensions 3 close to each other to curve the elastic member 5 together with the blade 2 and by putting the convex surface produced on the blade 2 in a portion-to-be-removed. In the top surface of the elastic member 5, a plurality of slits 8, 9 and 10 are provided, and a plurality of projections 11 are provided between the slits 8, 9 and 10 so that contact parts 11a of the projections 11 are brought close to and into contact with each other via the slits 8, 9 and 10 when the elastic member 5 is curved. The contact parts 11a function as a curvature restricting part for restricting the curvature of the elastic member 5 or a flexural rigidity changing part for changing the flexural rigidity of the elastic member 5.

### BACKGROUND OF THE INVENTION

The present invention relates to an excision instrument for removing tissues on the skin surface. More particularly, the present invention relates to an excision instrument that is used by bringing gripping extensions provided at both ends of an elastic member having a blade close to each other to curve the elastic member between both gripping extensions and by putting the convex surface of the concave surface and convex surface produced on the elastic member in a portion-to-be-removed.

Conventionally, an excision instrument of this type is used to excise tissues such as skin lesions and moles as described, for example, in U.S. Pat. No. 5,628,759.

For the excision instrument described in the above-described document, the user can curve the elastic member freely with a predetermined flexural rigidity. However, it is difficult to use the elastic member while surely keeping it in a state of having a desired curvature degree or flexural rigidity, which poses a problem in that the portion-to-be-removed cannot be excised stably using the blade.

### SUMMARY OF THE INVENTION

An objective of the present invention is to provide an easy-to-use excision instrument by improving the shape of an elastic member.

To achieve the foregoing objective and in accordance with a first aspect of the present invention, an excision instrument an elastic member and gripping extensions is provided. The elastic member has both ends and a blade edge. The gripping extensions are provided at both ends of the elastic member. The excision instrument is used by bringing the gripping extensions close to each other to curve the elastic member together with the blade edge and by putting the convex surface of the concave surface and convex surface produced on the elastic member on a portion-to-be-removed. The elastic member has a top surface that forms the concave surface when the elastic member is curved, and a curvature restricting part for restricting the curvature of the elastic member is provided on the top surface.

In accordance with a second aspect of the present invention, an excision instrument having an elastic member, a pair of gripping extensions, and a blade is provided. The elastic member has both ends. The gripping extensions are provided at both ends of the elastic member. The blade is mounted on the elastic member, and has a knife edge extending along a direction that connects gripping extensions to each other. The excision instrument is used by bringing the gripping extensions close to each other to curve the elastic member together with the blade edge and by putting the convex surface of the concave surface and convex surface produced on the elastic member in a portion-to-be-removed. The elastic member has a top surface that forms the concave surface when the elastic member is curved, and a curvature restricting part for restricting the curvature of the elastic member is provided on the top surface.

In accordance with a third aspect of the present invention, an excision instrument having an elastic member and gripping extensions is provided. The elastic member has both ends and a blade edge. The gripping extensions are provided at both ends of the elastic member. The excision instrument is used by bringing the gripping extensions close to each other to curve the elastic member together with the blade edge and by putting the convex surface of the concave surface and convex surface produced on the elastic member on a portion-to-be-removed. The elastic member has a flexural rigidity changing part that gives a plurality of different flexural rigidities to the elastic member.

### BRIEF DESCRIPTION OF THE DRAWINGS

FIG. 1(a) is an exploded perspective view of an excision instrument in accordance with an embodiment of the present invention;

FIG. 1(b) is a front view showing a state in which the excision instrument is being used;

FIGS. 2(a) and 2(b) are front views showing other states in which the excision instrument is being used;

FIGS. 3(a) and 3(b) are perspective views showing an assembled state of an excision instrument in accordance with the embodiment of FIGS. 1 to 2(b);

FIG. 4(a) is a plan view of the excision instrument;

FIG. 4(b) is a bottom view of the excision instrument;

FIG. 5(a) is a front view of the excision instrument; and

FIG. 5(b) is a rear view of the excision instrument.

### DETAILED DESCRIPTION OF THE PREFERRED EMBODIMENT

An excision instrument in accordance with one embodiment of the present invention will now be described with reference to the accompanying drawings.

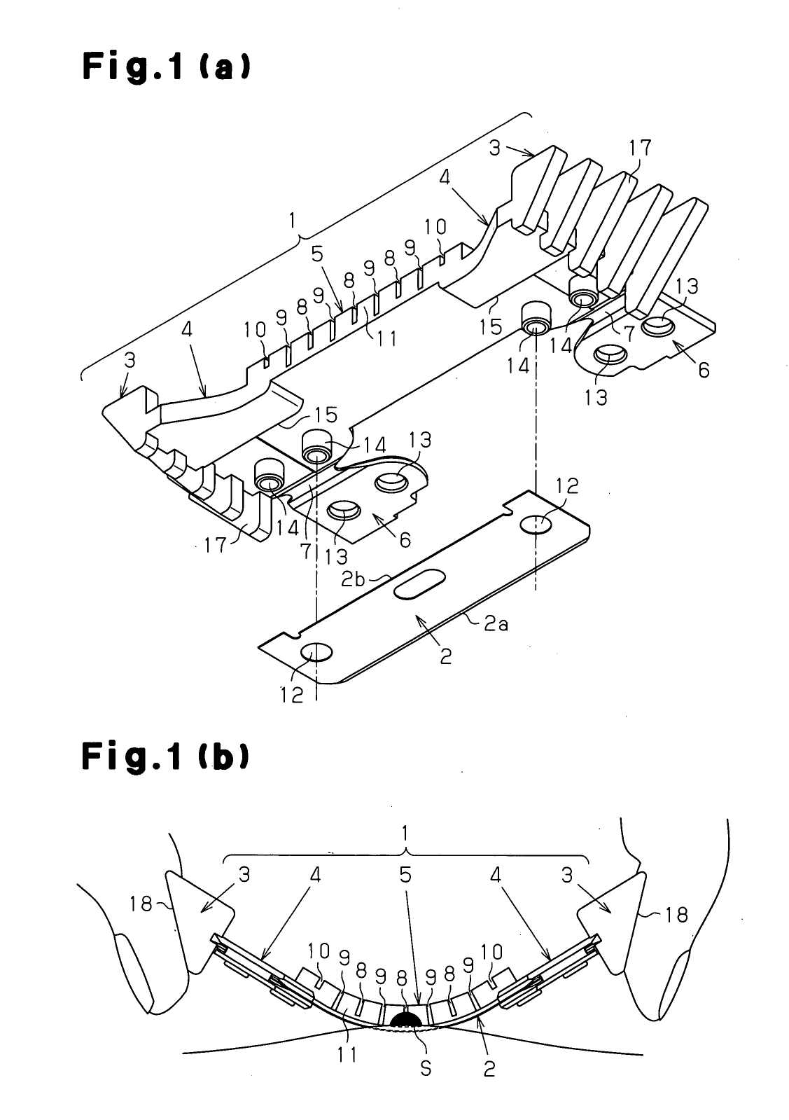

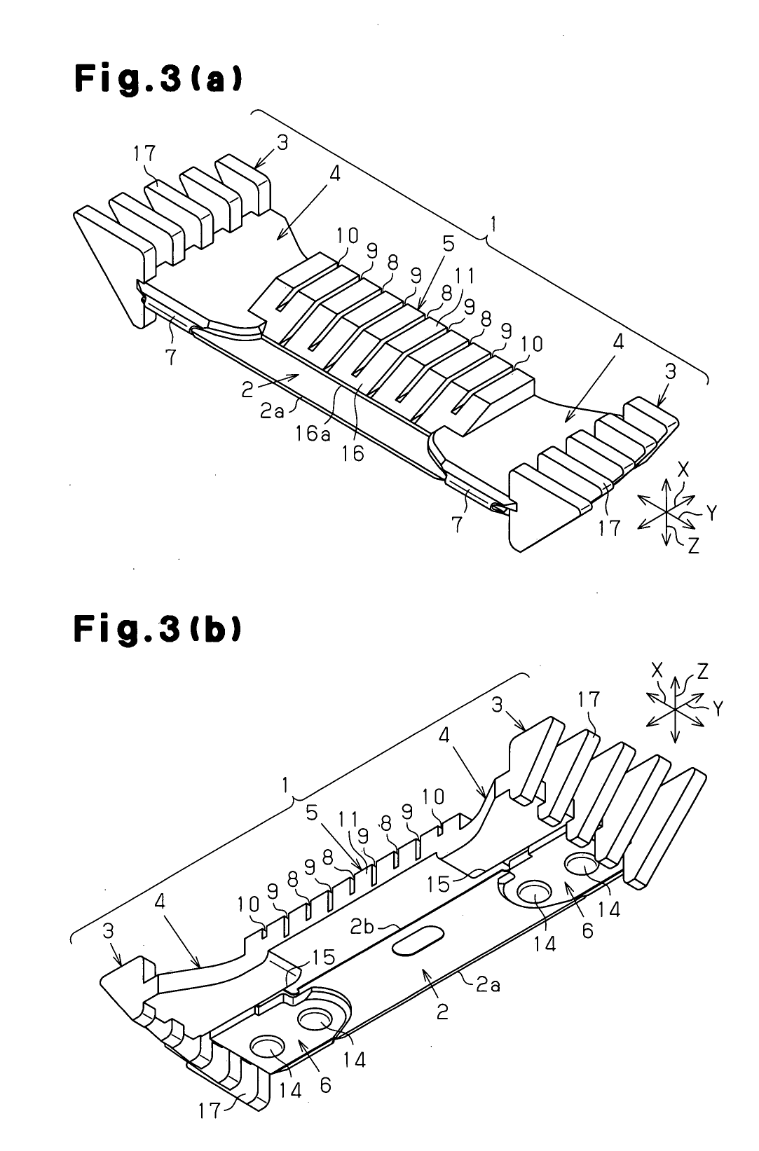

As shown in FIG. 1(a), this excision instrument includes an excision instrument body 1 integrally molded from polypropylene resin by injection molding and a blade 2 (0.1 mm thick) formed of stainless steel. In this excision instrument body 1, as shown in FIGS. 3(a) and 3(b), FIGS. 4(a) and 4(b), and FIGS. 5(a) and 5(b), an elastic member 5 is provided in the center of the body 1, support arms 4 are provided on both sides in the right and left direction Y of the elastic member 5, and gripping extensions 3 are connected to the body 1 via the support arms 4. In each support arm 4, in the end edge portion on the front side of the end edge portions on both sides in the front and rear direction X, press parts 6 are formed via bent parts 7. Since the excision instrument body 1 is integrally molded, the gripping extensions 3, the support arms 4, the elastic member 5, and the press parts 6 are molded easily.

In the elastic member 5, a plurality of slits 8, 9 and 10 having different depths D8, D9 and D10 (D9>D8>D10), respectively, are provided in the top surface on the upper side of both side surfaces in the upper and lower direction Z. The slits 8, 9 and 10 extend in parallel along the front and rear direction and are arranged at intervals in the right and left direction Y. Between these slits 8, 9 and 10, projections 11 are formed. One slit 8 having the depth D8 (2.0 mm) is arranged in the central portion in the right and left direction Y of the elastic member 5. Also, the slits 8 and the slits 9 each having the depth D9 (2.5 mm) are arranged alternately along the right and left direction Y, and the paired slits 10 each having the depth D10 (1.2 mm) are arranged in both end portions in the right and left direction Y of the elastic member 5. In these slits 8, 9 and 10, width dimensions W8, W9 and W9 in the right and left direction Y of the elastic member 5 are equal (each 0.6 mm), and are set smaller than the width dimension W11 in the right and left direction Y of each of the projections 11 (W11=2.4 mm>W8, W9, W10).

In the elastic member 5, height dimensions from the back surface on the lower side of both side surfaces in the upper and lower direction Z to the bottom surfaces of the slits 8, 9 and 10 are represented by H8, H9 and H10, respectively, and the height dimension from the back surface to the top surface of each of the projections 11 is represented by H11. The height dimensions H8, H9 and H10 (H9=0.5 mm<H8=1.0 mm<H10=1.8 mm) of the slits 8, 9 and 10 are set smaller than the height dimension H11 of the projection (H11=3.0 mm>H8, H9, H10). On the top surface of each of the projections 11, a contact part 11a is formed in the end edge portions on both sides in the right and left direction Y.

In the blade 2, a blade edge 2a extends in the right and left direction Y on the front side of both sides in the front and rear direction X, and a back edge 2b extends in the right and left direction Y on the rear side. Between the blade edge 2a and the back edge 2b, support holes 12 are formed in both end portions at the right and left of the blade 2.

In each of the right and left press parts 6, a pair of fastening holes 13 are formed. On the back surface of each of the support arms 4, a pair of fastening shafts 14 are formed, and at the rear of each of the fastening shafts 14, a step part 15 is formed. The blade 2 is put on the back surfaces of the support arms 4, the support holes 12 in the blade 2 are each fitted on one of the right fastening shaft 14 and the left fastening shaft 14, and the press parts 6 are bent by the bent parts 7 to press fit the fastening holes 13 to the fastening shafts 14. Accordingly, the blade 2 is held between the back surfaces of the support arms 4 and the press parts 6 so as to be incapable of being replaced. At this time, a gap G is produced between the back edge 2b of the blade 2 and the step part 15, so that the back edge 2b of the blade 2 does not come into contact with the step part 15. The press parts 6 may be made capable of being opened and closed with respect to the back surfaces the support arms 4 so that the blade 2 becomes replaceable.

On the top surface of the elastic member 5, an end edge part 16a is formed. The end edge part 16a is located adjacent to the blade edge 2a of the blade 2, and is in contact with the blade 2. From the end edge part 16a, a guide surface 16 is formed which tilts upward so as to be separate from the blade 2 in the direction from the blade edge 2a toward the back edge 2b of the blade 2.

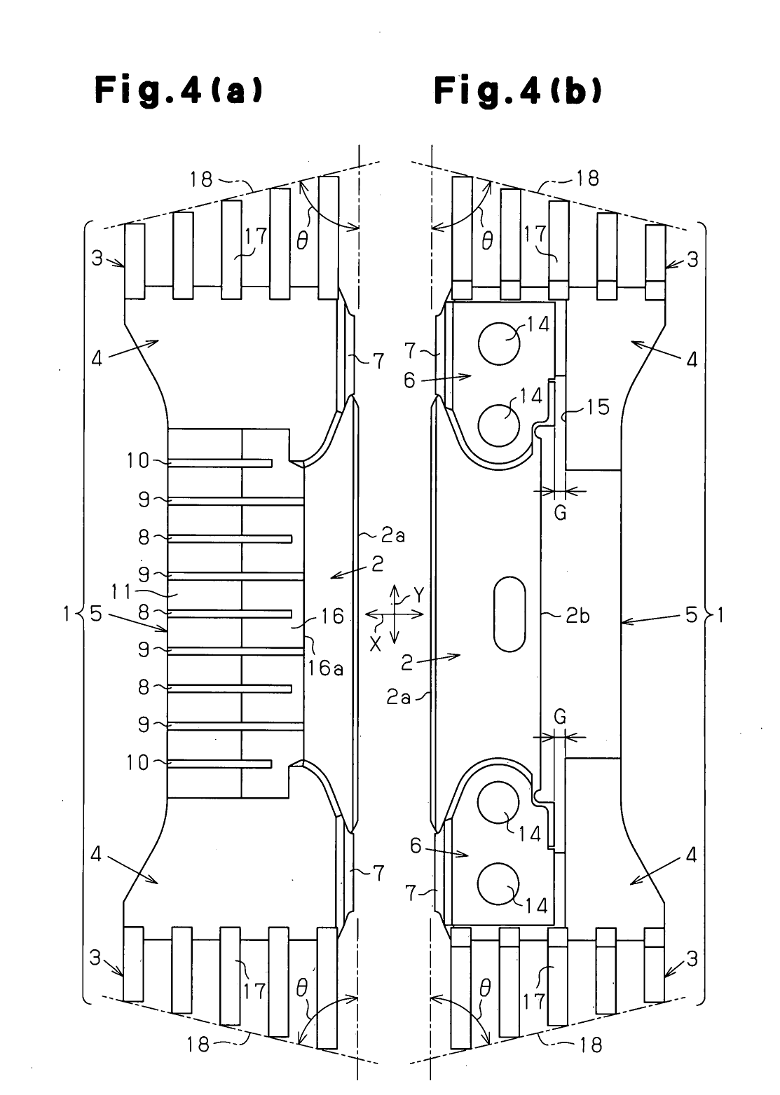

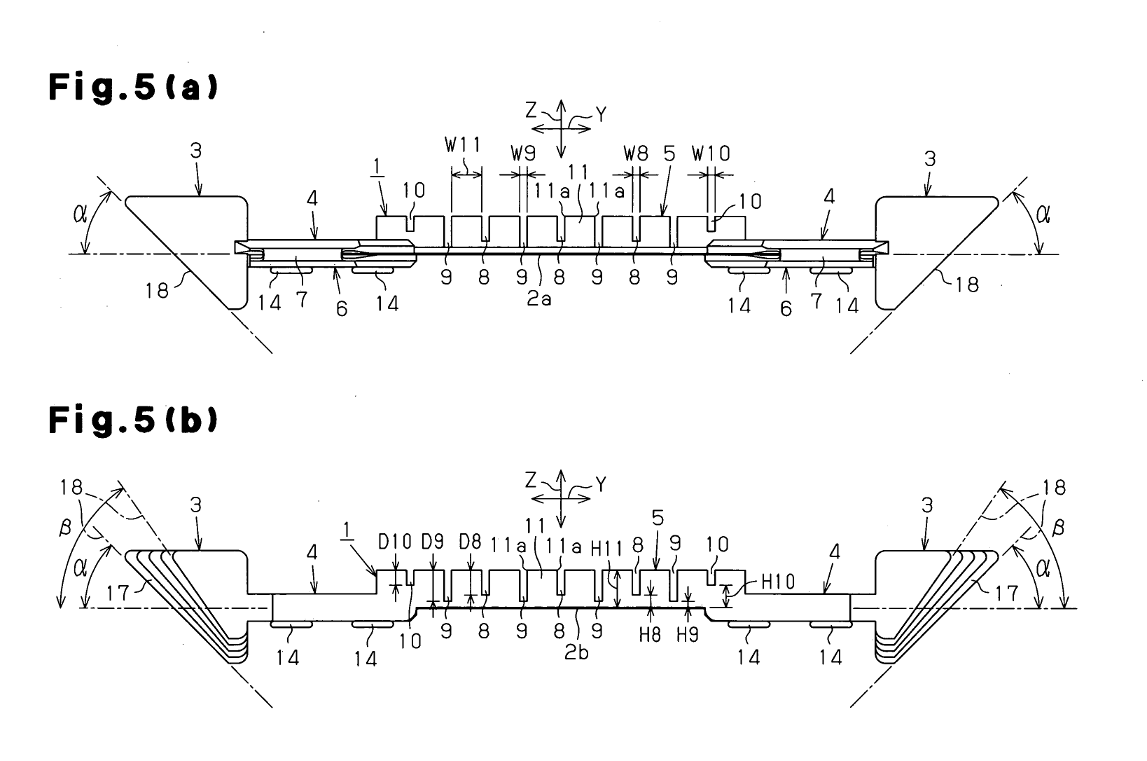

In each of the gripping extensions 3, a plurality of finger contact plate parts 17 are arranged with a gap provided therebetween. The outside edges of the finger contact plate parts 17 are arranged on a finger contact surface 18. Regarding the finger contact surface 18, the gaps between the finger contact plate parts 17 perform an antiskid function. As shown in FIGS. 5(a) and 5(b), the finger contact surface 18 tilts so as to be close to the elastic member 5 in the direction from the top surface to the back surface of the elastic member 5. Specifically, the finger contact surface 18 tilts at a tilt angle α (about 45°) on the side of the blade edge 2a with respect to the extending direction of the blade edge 2a (the right and left direction Y), and tilts at a tilt angle β (about 50°) on the side of the back edge 2b. The tilt angle of the finger contact plate part 17 increases gradually from the side of the blade edge 2a toward the side of the back edge 2b. Also, as shown in FIGS. 4(a) and 4(b), the finger contact surface 18 tilts at an angle θ (about 75°) with respect to the extending direction of the blade edge 2a (the right and left direction Y) so as to be close to the elastic member 5 in the direction from the blade edge 2a toward the back edge 2b of the blade 2.

For this excision instrument, the dimension in the front and rear direction X is set at about 20 mm, the dimension in the right and left direction Y is set at about 70 mm, and the dimension in the up and down direction Z is set at about 10 mm.

As shown in FIG. 1(b), this excision instrument is used by bringing the gripping extensions 3 close to each other to curve the elastic member 5 together with the blade 2 and by putting the convex surface of the concave surface and convex surface produced on the blade 2 in a portion-to-be-removed S (for example, a skin protrusion).

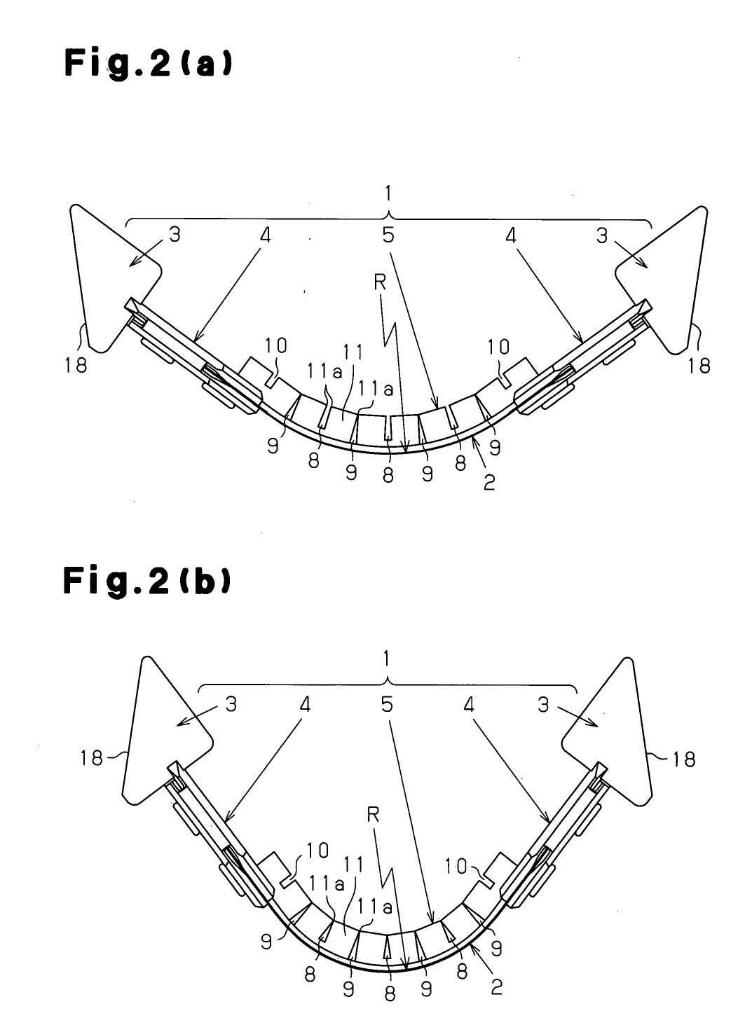

When the blade 2 is curved in this manner, the portion-to-be-removed S is excised easily to a desired size and depth, and also the back surface of the excision instrument and the user's finger do not come into contact with the portion-to-be-removed S and therefore do not become a hindrance when the excision instrument is used. Also, since a portion in which the;dimension in the right and left direction Y is at a maximum in the upper end portion of the gripping extension 3 is located above the blade 2, when the gripping extensions 3 are brought close to each other, the elastic member 5 and the blade 2 are bent downward and are bent in the reverse direction (upward). When the elastic member 5 and the blade 2 are flexed, as shown in FIG. 2(a), first, the contact parts 11a of the projections 11 that are adjacent to each other with the slit 9 having the depth D9 (D9>DB>D10) of the slits 8, 9 and 10 in the elastic member 5 being provided therebetween are brought into contact with each other, by which the curvature of the elastic member 5 is restricted at a radius of curvature R (about 25 mm), and the flexural rigidity of the elastic member 5 is made higher than the case shown in FIG. 1(b).

When the gripping extensions 3 are brought close to each other with a further greater force, as shown in FIG. 2(b), the contact parts 11a of the projections 11 that are adjacent to each other with the slit 8 having the depth D8 (D8>D10) provided therebetween are brought into contact with each other, by which the curvature of the elastic member 5 is restricted at a radius of curvature R (about 15 mm), and the flexural rigidity of the elastic member 5 is made further higher than the case shown in FIG. 2(a). That is to say, although the elastic member 5 can be curved with a small force at the early stage of curving, if the curvature degree increases, a force larger than that at the early stage of curving is required, so that the elastic member 5 are held with a desired curvature degree.

In this embodiment, therefore, the elastic member 5 can be held with a desired curvature degree or flexural rigidity by a curvature restricting part 11a, so that the portion-to-be-removed S is excised stably by the blade edge 2a, and also the elastic member 5 is prevented from being damaged.

(1) As described above, the excision instrument of this embodiment, having the elastic member 5 provided between the gripping extensions 3 and the blade edge 2a extending along the direction Y, which connects the gripping extensions 3 to each other, is used by bringing the gripping extensions 3 close to each other to curve the elastic member 5 together with the blade edge 2a and by putting the convex surface of the concave surface and convex surface produced on the elastic member 5 on the portion-to-be-removed S. On the surface of the elastic member 5, which forms the concave surface when the elastic member 5 is curved, one or more curvature restricting parts 11a for restricting the curvature are provided. The radius of curvature R of the elastic member 5 differs depending on the number of the curvature restricting parts 11a that come into contact with each other.

According to this configuration, the elastic member 5 can be held with a desired curvature degree by the curvature restricting part 11a, so that the portion-to-be-removed S is excised stably by the blade edge 2a.

(2) Also, the excision instrument of this embodiment, in which the blade 2 is attached to the elastic member 5 provided between the gripping extensions 3 and the blade edge 2a extending along the direction Y, which connects the gripping extensions 3 to each other, is provided on the blade 2, is used by bringing the gripping extensions 3 close to each other to curve the elastic member 5 together with the blade edge 2a and by putting the convex surface of the concave surface and convex surface produced on the blade 2 on the portion-to-be-removed S. On the top surface of the elastic member 5, which forms the concave surface when the elastic member 5 is curved, one or more curvature restricting parts 11a for restricting the curvature are provided. The radius of curvature R of the elastic member 5 differs depending on the number of the curvature restricting parts 11a that come into contact with each other.

In this case, the elastic member 5 can be held with a desired curvature degree by the curvature restricting part 11a, so that the portion-to-be-removed S is excised stably by the blade edge 2a.

(3) The curvature restricting part 11a is a flexural rigidity changing part 11a for changing the flexural rigidity of the elastic member 5. In this case, a plurality of different flexural rigidities are given to the elastic member 5. By the flexural rigidity changing part 11a, the elastic member 5 is held with a desired curvature degree and flexural rigidity, so that the portion-to-be-removed S is excised more stably by the blade edge 2a.

(4) The curvature restricting part is the contact parts 11a. The curvature restricting part thus has a simple structure.

(5) On the top surface of the elastic member 5, the projections 11 are provided, and the contact parts 11a are provided on each of the projections 11. The curvature restricting thus has a simple structure.

(6) In the top surface of the elastic member 5, the slits 8, 9 and 10 are arranged along the direction Y, which connects the gripping extensions 3 to each other, and the projections 11 are provided between the slits 8 and 9 and between the slits 9 and 10 so that the contact parts 11a of the projections 11 are brought close to each other via the slits 8, 9 and 10 therebetween and come into contact with each other when the elastic member 5 is curved. The curvature restricting part thus has a simple structure.

(7) The excision instrument of this embodiment, having the elastic member 5 provided between the gripping extensions 3 and the blade edge 2a extending along the direction Y, which connects the gripping extensions 3 to each other, is used by bringing the gripping extensions 3 close to each other to curve the elastic member 5 together with the blade edge 2a and by putting the convex surface of the concave surface and convex surface produced on the elastic member 5 on the portion-to-be-removed S. The elastic member 5 is provided with the flexural rigidity changing part 11a that gives a plurality of different flexural rigidities. The flexural rigidity increases stepwise as the radius of curvature R of the elastic member 5 decreases.

In this case, the elastic member 5 is held with a desired flexural rigidity by the rigidity changing part 11a, so that the portion-to-be-removed S is excised more stably by the blade edge 2a.

(8) The excision instrument of this embodiment, having the elastic member 5 provided between the gripping extensions 3 and the blade edge 2a extending along the direction Y, which connects the gripping extensions 3 to each other, is used by bringing the gripping extensions 3 close to each other to curve the elastic member 5 together with the blade edge 2a and by putting the convex surface of the concave surface and convex surface produced on the elastic member 5 on the portion-to-be-removed S. In the top surface of the elastic member 5, the slits 8, 9 and 10 are arranged along the direction Y, which connects the gripping extensions 3 to each other, and the projections 11 are provided between the slits 8 and 9 and between the slits 9 and 10. The width dimensions W8, W9 and W10 of the slits 8, 9 and 10 are set smaller than the width dimension W11 of each of the projections 11 in the direction Y, which connects the gripping extensions 3 to each other, and the height dimensions H8, H9 and H10 of the bottom surfaces of the slits 8, 9 and 10 are set smaller than the height dimension H11 of the top surface of each of the projections 11 with respect to the back surface of the elastic member 5 on the opposite side to the slits 8, 9 and 10.

In this case, even if the elastic member 5 is made thick, the flexural rigidity of the elastic member 5 is decreased by the slits 8, 9 and 10, by which the elastic member 5 is bent easily, and therefore the portion-to-be-removed S is excised easily by the blade edge 2a.

(9) In the top surface of the elastic member 5, the slits 8, 9 and 10 having the different depths D8, D9 and D10, respectively, are arranged in combination. By changing the depths D8, D9 and D10 of the slits 8, 9 and 10, the curvature restricting part 11a and the flexural rigidity is changed easily.

(10) On the mounting members 4 and 5, including the elastic member 5, provided between the gripping extensions 3, the press parts 6 are provided via the bent parts 7, and the blade 2 is held between the back surfaces of the mounting members 4 and 5, including the elastic member 5, on which the blade 2 is put and the press parts 6. In this case, the blade 2 is attached easily to the mounting members 4 and 5, including the elastic member 5.

(11) The finger contact surface 18 on the outside of both surfaces along the direction Y, which connects the gripping extensions 3 to each other, tilts with respect to the extending direction Y of the blade edge 2a so as to be close to the elastic member 5 in the direction from the top surface of the elastic member 5 toward the back surface of the elastic member 5, which is on the opposite side to the top surface thereof. In this case, by simply putting the user's fingers on the finger contact surfaces 18 of the gripping extensions 3, the gripping extensions 3 are brought close to each other and thereby the elastic member 5 is bent easily.

(12) The finger contact surface 18 on the outside of both surfaces along the direction Y, which connects the gripping extensions 3 to each other, tilts with respect to the extending direction Y of the blade edge 2a so as to be close to the elastic member 5 in the direction from the blade edge 2a toward the opposite side to the blade edge 2a. In this case, when the excision instrument is used by putting the blade edge 2a on the portion-to-be-removed S, the finger that is put on the finger contact surface 18 of the gripping extension 3 is less liable to slip.

(13) On the top surface of the elastic member 5, the guide surface 16 extending from the end edge part 16a that is located adjacent to the blade edge 2a of the blade 2 and is in contact with the blade 2 is provided. The guide surface 16 tilts so as to be separate from the blade 2 in the direction from the blade edge 2a of the blade 2 toward the opposite side to the blade edge 2a. In this case, excision chips of the portion-to-be-removed S is removed properly by the guide surface 16 extending from the end edge part 16a on the blade 2.

The excision instrument may be configured as described below in addition to the above-described embodiment.

In the above-described embodiment, the blade 2 is mounted on the back surface of the elastic member 5. However, the configuration may be such that the blade 2 itself is made an elastic member, and on the top surface of the blade 2, the curvature restricting part or the flexural rigidity changing part is provided in each of the slits.

In the above-described embodiment, the first-stage curvature restricting part (contact part 11a) using the slit 9 and the second-stage curvature restricting part (contact part 11a) using the slit 8 are provided. However, only one curvature restricting part or three or more curvature restricting parts may be provided.

In the above-described embodiment, by changing the depths D8, D9 and D10 of the slits 8, 9 and 10 with the width dimensions W8, W9 and W10 thereof being equal, the timing of bringing the contact parts 11a into contact with each other is shifted to produce two-stage curvature restricting part. However, the timing of bringing the contact parts 11a into contact with each other may be shifted by changing the width dimensions W8, W9 and W10 of the slits 8, 9 and 10 with the depths D8, D9 and D10 thereof being equal.

In the above-described embodiment, the slits 8, 9 and 10 are arranged in parallel with each other in the right and left direction Y. However, the arrangement, the shape, and the number of the slits 8, 9 and 10 may be changed.

The curvature restricting part or the flexural rigidity changing part may be provided by variously changing the shapes of the projections provided on the top surface of the elastic member 5 and by utilizing the contact parts lying in the projections.

The slits 8, 9 and 10 may be filled with an elastic body.

On both sides in the up and down direction Z, the slits 8, 9 and 10 may be provided on the elastic member 5. In this case, the whole of the excision instrument may be of a symmetrical shape on both sides in the up and down direction Z, the whole of the excision instrument may be of an asymmetrical shape on both sides in the up and down direction Z, or the curvature restricting part or the flexural rigidity changing part may be provided in the slits 8, 9 and 10 on both of the upper and lower sides or in the slits 8, 9 and 10 on one side.

In the above-described embodiment, the whole of the excision instrument is of a symmetrical shape on both sides in the right and left direction Y. However, the whole of the excision instrument may be of an asymmetrical shape on both sides in the right and left direction Y.

The blade edge 2a of the blade 2 may be of a wavy shape in which recessed parts recessed in the front and rear direction X and projecting parts projecting in the front and rear direction X are arranged alternately in the right and left direction Y, may be of a wavy shape in which recessed parts recessed in the up and down direction Z and projecting parts projecting in the up and down direction Z are arranged alternately in the right and left direction Y, may be of a recessed curved shape recessed in the front and rear direction X or a projecting curved shape projecting in the front and rear direction X, or may be provided with a change in thickness in the up and down direction Z.

The finger contact surface 18 of the gripping extension 3 may be curved into a concave shape or a convex shape.

1. An excision instrument comprising:
an elastic member having both ends and a blade edge; and
gripping extensions provided at both ends of the elastic member, wherein the excision instrument is used by bringing the gripping extensions close to each other to curve the elastic member together with the blade edge and by putting the convex surface of the concave surface and convex surface produced on the elastic member on a portion-to-be-removed,
wherein the elastic member has a top surface that forms the concave surface when the elastic member is curved, and a curvature restricting part for restricting the curvature of the elastic member is provided on the top surface.

2. The excision instrument according to claim 1, wherein the curvature restricting part is also a flexural rigidity changing part for changing the flexural rigidity of the elastic member.

3. The excision instrument according to claim 1, wherein the curvature restricting part is a plurality of contact parts that come into contact with each other when the elastic member is curved and restricts the curvature of the elastic member.

4. The excision instrument according to claim 3, wherein a projection is provided on the surface of the elastic member, and the contact parts are provided on the projection.

5. An excision instrument comprising:
an elastic member having both ends;
a pair of gripping extensions provided at both ends of the elastic member; and
a blade mounted on the elastic member, the blade having a knife edge extending along a direction that connects gripping extensions to each other,
wherein the excision instrument is used by bringing the gripping extensions close to each other to curve the elastic member together with the blade edge and by putting the convex surface of the concave surface and convex surface produced on the elastic member in a portion-to-be-removed, and
wherein the elastic member has a top surface that forms the concave surface when the elastic member is curved, and a curvature restricting part for restricting the curvature of the elastic member is provided on the top surface.

6. The excision instrument according to claim 5, wherein the curvature restricting part is also a flexural rigidity changing part for changing the flexural rigidity of the elastic member.

7. The excision instrument according to claim 5, wherein the curvature restricting part is a plurality of contact parts that come into contact with each other to restrict the curvature of the elastic member when the elastic member is curved.

8. The excision instrument according to claim 7, wherein a plurality of projections are provided on the top surface of the elastic member, and the contact parts are provided on the projections.

9. The excision instrument according to claim 8, wherein a plurality of slits are provided along the direction that connects the gripping extensions to each other in the top surface of the elastic member, the projections are provided between an adjacent pair of the slits, and when the elastic member is curved, the contact parts of the projections are brought close to and into contact with each other via the slits therebetween.

10. The excision instrument according to claim 9, wherein the width dimension of each of the slits is set smaller than the width dimension of each projection along a direction that connects the gripping extensions of the projection to each other, and the height dimension from the back surface of the elastic member to the bottom surface of each of the slits is set smaller than the height dimension from the back surface of the elastic member to the top surface of each of the projections.

11. An excision instrument comprising:
an elastic member having both ends and a blade edge; and
gripping extensions provided at both ends of the elastic member, wherein the excision instrument is used by bringing the gripping extensions close to each other to curve the elastic member together with the blade edge and by putting the convex surface of the concave surface and convex surface produced on the elastic member on a portion-to-be-removed,
wherein the elastic member has a flexural rigidity changing part that gives a plurality of different flexural rigidities to the elastic member.
